(19) United States
(12) Patent Application Publication
(10) Pub. No.: US 2005/0118208 A1
(43) Pub. Date: Jun. 2, 2005

(54) Powder formulation comprising conjugated octadecapolyenic acids

(75) Inventors: Wolfgang Bewert, Frankenthal (DE); Morton Mohr Hansen, Alleroed (DK); Oliver Hasselwander, Landau (DE); Ulrike Sindel, Muenchen (DE); Arne Ptock, Ludwigshafen (DE); Willy Hinz, Mannheim (DE); Angelika-Maria Pfeiffer, Birkenheide (DE); Andreas Habich, Speyer (DE); Bruno Kaesler, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/495,754

(22) PCT Filed: Nov. 27, 2002
(86) PCT No.: PCT/EP02/13345

(30) Foreign Application Priority Data
Nov. 27, 2001 (DE) 101 58 046.0

(51) Int. Cl. A61K 31/202; A61K 7/00

(52) U.S. Cl. 424/401; 514/560

(57) ABSTRACT

The present invention relates to pulverulent formulations comprising at least one conjugated trans/-cis-octadecapolyenoic acid and also production and use thereof for use in foods, food supplements, feedstuffs, feed additives, pharmaceutical and cosmetic preparations.

The present invention relates to a formulation for use in foods, food supplements, feedstuffs, feed additives, pharmaceutical and cosmetic preparations and also preparation and use thereof.

Fatty acids have a multiplicity of uses in the food industry, animal nutrition, cosmetics and the pharmaceutical sector. Depending on whether free saturated or unsaturated fatty acids, or triglycerides having an elevated content of saturated or unsaturated fatty acids are concerned, they are suitable for the most varied applications. Thus a high proportion of lipids containing unsaturated fatty acids and especially containing polyunsaturated fatty acids, are important for animal and human nutrition, since these, furthermore, have a beneficial effect on the triglyceride level and cholesterol level and thus reduce the risk of heart disease. Unsaturated fatty acids are used in various dietetic foods or medicaments. Polyunsaturated fatty acids are essential nutrients, since they cannot be synthesized by humans and animals themselves.

Particularly valuable unsaturated fatty acids are the conjugated polyunsaturated fatty acids, for example conjugated linoleic acid (CLA). Conjugated polyunsaturated fatty acids are somewhat rare compared with other polyunsaturated fatty acids.

CLA is a collective term for positional and structural isomers of linoleic acid which are distinguished by a conjugated double bond system starting at carbon atom 7, 8, 9, 10 or 11. Geometric isomers, that is to say cis-cis, trans-cis, cis-trans, trans-trans, exist for each of these positional isomers.

Especially C18:2 cis-9, trans-11 and C18:2 trans-10, cis-12 CLAs, which are the most biologically active isomers, are of particular interest, since they have been found to be cancer-preventive in animal experiments, act in an anti-arteriosclerotic manner, and, in humans and animals, reduce the body fat content. Commercially, CLAs are chiefly marketed in the form of their free fatty acids.

For humans, the most important natural sources of CLAs are especially animal fats. Thus fats of ruminant animals, such as cattle (Chin. Journal of Food Composition and Analysis, 5, 1992: 185-197) and sheep, and also dairy products have very high CLA concentrations. In cattle, from 2.9 to 8.9 mg of CLA/g of fat are found. In contrast, vegetable oils, margarines and fats of non-ruminant animals have CLA concentrations of only from 0.6 to 0.9 mg/g of fat.

For CLAs, a number of beneficial effects have been demonstrated. For instance, administering conjugated linoleic acid reduces the body fat of humans and animals, and improves the feed utilization in animals (WO 94/16690, WO 96/06605, WO 97/46230, WO 97/46118). Administering conjugated linoleic acid also beneficially affects, for example, allergies (WO 97/32008), diabetes (WO 99/29317) or cancer (Banni, Carcinogenesis, Vol. 20, 1999: 1019-1024, Thompson, Cancer, Res., Vol. 57, 1997: 5067-5072). Polyunsaturated fatty acids are also added to baby food to “increase the nutritional value” and as essential building blocks which promote growth and brain development.

Since CLA occurs naturally in significant amounts only in ruminants and products thereof, such as milk, cheese, etc., there is a great need for alternative sources for the CLA originating from these animal sources to ensure balanced and healthy nutrition, in particular when the supply of animal fats is reduced and/or is inadequate.

Commercially, CLAs are principally marketed as free fatty acids and also fatty esters. Fatty acids such as CLA generally do not occur in nature as free fatty acids, but are esterified to form triglycerides. In addition free fatty acids frequently have an odor perceived as unpleasant and are not well tolerated.

WO 97/37546 discloses mixtures of non-hardened and hardened fats having particle sizes from 20 to 50 μm.

WO 01/78531 additionally discloses compositions which comprise more than 20% of pulverulent conjugated linoleic acids.

A disadvantage of the octadecapolyenoic acids used hitherto, for example CLAs, is that these are insufficiently stable for relatively longterm storage.

It is thus an object of the present invention to provide formulations of octadecapolyenoic acids which have improved stability and tabletability. In particular, pulverulent formulations for use in foods, food supplements, feedstuffs, feed additives, pharmaceutical and cosmetic preparations comprising at least one conjugated trans/cis octadecapolyenoic acid having said properties are to be provided.

We have found that this object is achieved by less than 5% of the fatty acid content being 11, 13-octadecadienoic acid isomers, 8-, 10-octadecadienoic acid isomers, cis-, cis-octadecadienoic acid isomers or trans-, trans-octadecadienoic acid isomers or mixtures of these isomers.

Very particularly preferably, less than 3% of the fatty acid content is 11-, 13-octadecadienoic acid isomers, 8-, 10-octadecadienoic acid isomers, cis-, cis-octadecadienoic acid isomers or trans, trans-octadecadienoic acid isomers or mixtures of these isomers. Very highly preferably, less than 1% of the fatty acid content is 11, 13 octadecadienoic acid isomers, 8, 10 octadecadienoic acid isomers, cis-, cis-octadecadienoic acid isomers or trans, trans-octadecadienoic acid isomers or mixtures of these isomers.

Preferably, as octadecapolyenoic acids, use is made of octadecadienoic acid and octadecatrienoic acid. These substances can be used individually or as mixtures, if appropriate with further fatty acids. In addition, other components can also be present. Octadecapolyenoic acids hereinafter means free acids or esters thereof, and also alkali metal salts and alkaline earth metal salts, in particular calcium salts.

According to the invention, particular preference is given to the use of octadecadienoic acid. This is taken to mean octadecadienoic acids in the form of their free acid or in the form of their esters, for example methyl, ethyl, propyl, buthyl esters.

A preferred embodiment of the invention provides that at least 50% of the fatty acid content is 9-cis-, 11-trans- and 10-trans-, 12-cis-octadecadienoic acids. Particular preference is given to at least 60% of the fatty acid content being 9-cis-, 11-trans- and 10-trans-, 12-cis-octadecadienoic acids. Very particular preference is given to contents of at least 70%.

As octadecatrienoic acid, use can be made of calendic acid, for example.

Calendic acid is a C18:3 fatty acid having a t8, t10, c12-configuration. It is thus a conjugated trans/cis octadecatrienoic acid. Calendic acid is the fatty acid responsible for the reduction in feed intake and improved feed utilization in mice when calendular oil is added to the feedstuff, as demonstrated by the comparative experiments in the examples using corn oil. The chemical preparation of calendic acid is described in U.S. Pat. No. 3,356,699. Calendic acid occurs naturally in Calendula officinalis, for example (Earle et al., Lipids, 1, 1964: 325-327, Takagi et al., Lipids, 17, 1981: 716-723, Ul'chenko et al., Lipids of Calendula officinalis, Chemistry of Natural Compounds, 34, 1998: 272-274). Biochemical investigations on the synthesis of calendic acid are known (Crombie et al., J. Chem. Soc. Chem. Commun., 15, 1984: 953-955 and J. Chem. Soc. Perkin Trans., 1, 1985: 2425-2434).

Other conjugated trans/cis octadecatrienoic acids can be obtained by chemical modification of linolenic-acid-containing oils, for example from linseed oil, soybean oil or hemp oil.

Examples of further octadecapolyenoic acids which can be used according to the invention are eleostearic acid or punicic acid.

In the inventive formulation, further unsaturated fatty acids may also be present.

The term “fatty acid” is taken to mean an unbranched carboxylic acid having an even number of carbon atoms and from 16 to 22 carbon atoms.

The octadecapolyenoic acid described can be prepared by processes known to those skilled in the art, for example by the process described in WO 99/47135. There, inter alia, the chemical synthesis of CLA alkyl esters and the enzymatic production of CLA triglyceride are described.

The conjugated octadecapolyenoic acids described can be prepared, for example by a single-stage isomerization of polyunsaturated free fatty acids, for example linoleic acid, which are esterified in the form of a glyceride, in particular a triglyceride, with catalysis by imino bases or iminophosphorane bases, in particular aminoimino bases or aminoiminophosphorane bases.

The term “glyceride” is taken to mean a glycerol esterified with one, two or three carboxylic acid radicals (mono-, di- or triglyceride). “Glyceride” is also taken to mean a mixture of different glycerides. The glyceride or the glyceride mixture can comprise other additives, for example free fatty acids, antioxidants, proteins, carbohydrates, vitamins and other substances, for example as listed hereinafter under “additives”.

The glyceride used can be a synthetic or naturally occurring glyceride oil or a derivative thereof. “Glyceride” is also taken to mean synthetic or naturally occurring fatty esters and/or glyceride-containing oils and fats. A “glyceride” is further taken to mean derivatives of glycerol. In addition to the abovedescribed fatty glycerides, these also include glycerophospholipids and glyceroglycolipids. Preference is given here to the glycerophospholipids such as lecithin (phosphatidylcholine), cardiolipin, phosphatidylglycerol, phosphatidylserine and alkylacylglycerophospholipids such as plasmalogen. In particular, this comprises derivatives in which the fatty acid composition of the naturally occurring non-conjugated or saturated glycerides has not significantly changed.

As starting materials, preference is given particularly to glycerides or mixtures of glycerides, in particular mono-, di- or triglycerides which are esterified with at least one, preferably two or three, polyunsaturated, in particular conjugated, fatty acids. Consequently, preference is given to synthetic or natural glycerides which contain acyl radicals having from 1 to 22 carbon atoms, preferably having 18 carbon atoms. Particular preference is given to natural oils and fats which contain polyunsaturated homoconjugated acyl radicals having more than 16 carbon atoms and fewer than 22 carbon atoms, preferably from 18 to 20 carbon atoms.

The term “oil” or “fat” is taken to mean a fatty acid mixture which comprises unsaturated, nonconjugated or conjugated esterified fatty acid(s), in particular linoleic acid. Preference is given to the oil or fat having a high content of unsaturated, nonconjugated esterified fatty acid(s), in particular linoleic acid. Preferably, the content of unsaturated, nonconjugated esterified fatty acids is approximately 30%, more preferably 50%, still more preferably 60%, 70%, 80%, 90% or more. For its determination, for example the content of fatty acid after conversion of the fatty acids to the methyl esters (by transesterification) can be determined by gas chromatography.

The oil or fat can comprise differing other saturated or unsaturated fatty acids, for example calendic acid, palmitic acid, stearic acid, oleic acid etc. In particular, the content of the various fatty acids in the oil or fat can fluctuate depending on the production process. Fatty esters are likewise comprised by the inventive formulation, in particular fatty esters which are formed on the production of oil from vegetable material. Preferably, the fatty esters are present as glyceride, in particular of triglyceride.

Vegetable or animal starting material which can be used according to the invention is, for example, olive oil, coconut oil, coconut fat, sesame oil, rice germ oil, bamboo oil, bamboo fat, sunflower oil, rapeseed oil, fish oil, soybean oil, palm oil, safflower oil, linseed oil, wheatgerm oil, peanut oil, cottonseed oil, corn germ oil, pig fat, beef fat, poultry fat, milk fat, tung oil, or shea oil or a derivative or a mixture thereof. Particular preference is given in particular to oils and fats which have a high content of linoleic acid, for example sunflower oil, soybean oil, cottonseed oil, corn or wheatgerm oil, safflower oil, thistle oil, rapeseed oil and also in particular oils or fats from modified plant varieties, in particular high linoleic acids, for example linola (from linseed oil). The modified plant varieties can be bred or can advantageously also be produced by mutagenesis (for example GMO) (Angew. Chem. 2000, 112, 2292-2310).

The starting material for preparing the inventive conjugated octadecapolyenoic acids can be produced by customary processes known to those skilled in the art, for example from plants. For example oil can be produced by pressing seeds, for example having a high hull content or from hulled seeds. For pressing and production, in addition to the plant seeds, use can also be made of other plant parts, for example leaves, tubors, stems, blossoms, fruits etc. of suitable plants which have a high content of unsaturated fatty acids, preferably esterified with triglycerides. Whole plants can also be used. The pressed material can also be pressed repeatedly.

Likewise suitable for producing inventively suitable oils and fats are microorganisms, such as Thraustochytrium or Schizochytrium strains, algae such as Phaeodactylum tricornutum or Crypthecodinium species, ciliates, such as Stylonychia or Colpidium, fungi, such as Mortierella, Entomorphthora or Mucor. By strain selection, a number of mutant strains of the corresponding microorganisms have been developed which produce a series of desirable compounds, including PUFAs and which are also suitable for producing said fatty acids or oils. In particular, microorganisms can be produced by suitable transformations, for example using desaturase- or elongase-coding nucleic acid molecules.

Preferably, for the purposes of the present invention, linoleic esters are converted to the conjugated linoleic acid ester (CLA).

Preference is therefore given to starting materials which contain linoleic esters, for example those which have a high content of linoleic-acid-containing triglycerides. Particular preference is therefore given to natural oils and fats which have a high content of linoleic acid, for example sunflower oil, soybean oil, safflower oil, linseed oil or derivatives thereof.

In a process variant for producing the inventive conjugated octadecanepolyenoic acids, the catalysis can be carried out using the compound I 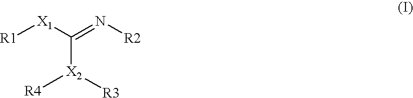
where independently of one another
X₁ can be —NH— or —PH—, preferably —NH—,
X₂ can be C—H, N, or P—, preferably N or P, most preferably N, and where
R1 to R4 independently of one another can be:
H,
branched or unbranched C₁- to C₂₀-alkyl, where from 1 to 3 carbon atoms can be replaced by O, S, NZ and/or
—X₃—(C═X₄)—;
mono-, bi-, or tricyclic, aromatic, saturated or partially unsaturated C₃- to C₆-alkyl carbocycle or heterocycle containing from 3 to 17 carbon atoms, where from 0 to 3 heteroatoms can be selected from O, S, NZ and/or —X₃—(C═X₄)—;
and where each carbon atom of the alkyl chains or of the ring can bear up to three of the following substituents OZ, SZ, (C═O)—OZ, NZZ₁, C₁- to C₆-alkyl;
where X₃ can be a bond, O, S, or NZ, and/or X₄ can be O, S, or NZ; and
where Z and/or Z₁ independently of one another can be H or C₁- to C₆-alkyl.
X₁ and X₂ can thus be part of a ring via R3 or R4,
in particular R1 and R4 and also R2 and R3 can be part of a ring.

The rings can consequently bear heteroatoms or else further double bonds. In particular, R1 and R4 and also R2 and R3 can be bonded in a cycle via ((CH)₂)ₙ where n=2,3,4,5.

It is known to those skilled in the art that it is possible to use compound I where
X₁ is —PH— only under inert conditions which prevent oxidation of —PH—. Compound I can also be present bound in polymer, for example as Merrifield resin, for example heterogenized as Merrifield resin on various chloromethylated poly(styrene/divinylbenzene) resins, or heterogenized on polystyrene resin after introducing a spacer in the form of an alkyl chain.

The catalyst can be present not only as pure substance, but also immobilized, for example bound to a polymer (polymer-bound guanidine bases (J. Mol. Catal. A: Chemical 109 (1996) 37-44; Pure Appl. Chem., A29(3), 249-261 (1992)), polymer-bound aminoiminophorane bases (Chimia 39 (1985) No.9, 269-272) or enclosed in a support (cyclohexylguanidine in zeolite Y; THL, Vol. 38, No. 8, 1325-1328, (1997)).

Compound I can have the following structure (Ia): 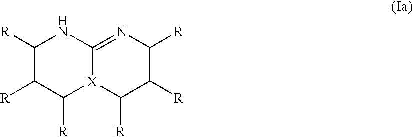
where X is C—H, N, or P, preferably N or P, most preferably N, and R is as above for R1 to R4.

Likewise suitable as catalyst are compounds (II): 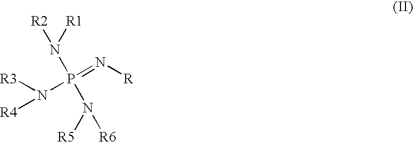
for example, the compounds (IIa) can be used: 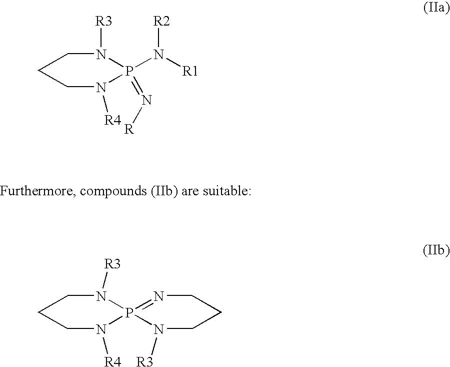
where, in the compounds (II) to (IIb) R and also R1 to R6 have the meanings given above for R1 to R4.

In particular, R1 and R2, R3 and R4 and/or R5 and R6 can be cyclically linked.

The compounds I or II, in particular imino bases or iminophosphazene bases, preferably aminoimino bases or aminoiminophosphazene bases, can catalyze the isomerization to nonconjugated polyunsaturated fatty acids in the glyceride without addition of protic solvents, for example alkyl alcohols. In particular, the isomerization can be carried out using guanidine bases for example as catalyst.

Likewise, 1,5,7-triazabicyclo[4.4.0.]dec-5-ene (TBD) or analogous diaza bases, for example 1,2,3,4,4a,5,6,7-octahydro-1,8-naphthyridine [Cas. 60832-40-8] can be used.

In addition, use can be made of the phosphazene bases phosphazene base P4-T-Bu [Cas. 111324-04-0], phosphazene base P1-T-Oct No. [Cas. 161118-69-0], phosphazene base P1-T-Bu-tris(tetramethylene) [Cas. 161118-67-8], phosphazene base P2-T-Bu [Cas. 111324-03-9], phosphazene base P4-T-Oct [Cas. 153136-05-1], the salts (1,1,1,3,3,3-hexakis(dimethylamino)diphosphazenium fluoride [Cas 137334-99-7], 1,1,1,3,3,3-hexakis(dimethylamino)diphosphazenium tetrafluorborate [Cas. 137334-98-6] or 2-tert-butylimino-2-diethylamino-1,3-dimethylperhydro-1,3,2-diaza-phosphorine [Cas. 98015-45-3]. Also usable are salts thereof (for example BF₄⁻, or F salt) or other salts of said compounds.

To prepare the inventive conjugated octadecapolyenoic acids, aprotic solvents can be used, for example ethyl acetate, hexane, heptane, DMSO, DMF, MTBE, or THF. However, use can also be made of protic solvents, for example alkyl alcohols. However, on the basis of the disclosure of the present invention, a person skilled in the art can also, by simple test series, find other solvents which can likewise be used.

In particular when use is made of the inventive formulations for foods, food supplements, feedstuffs, feed additives, cosmetic or pharmaceutical preparations, it is advantageous to prepare the conjugated octadecapolyenoic acids using solvents which are not toxic, or are as little toxic as possible, so that it is not necessary to separate off the solvent after carrying out the process, or that any slight traces which may remain are not damaging to the desired use.

Depending on the use, another solvent can be considered advantageous. For example, protic solvents, for example methanol, ethanol, etc., can be used, for example for the conjugation of unsaturated homoconjugated fatty esters. It can be advantageous to use a solvent which can be readily removed from the fatty acids, glycerides, oils or fats, for example may be extracted by taking in water, or which is low-boiling, for example MTBE.

If appropriate, the inventive conjugated octadecapolyenoic acids can be prepared without solvent. The reaction temperature of the process must then be adapted in such a manner that the melting point of the catalyst used is exceeded. It can be advantageous to carry out the process at low temperatures and for this to dissolve the catalyst in a suitable solvent.

The temperature can be chosen depending on solvent, catalyst, pressure and starting material, but should be below 180° C., however. Higher temperatures in the isomerization can lead to lower yields, for example owing to derivatizations or destruction of the fatty acids.

The temperature and reaction time also depend, for example, on the strength of the base used. For instance, TBD is a strong guanidine base, but the reaction temperature, owing to the high melting point, must be selected above 130° C. if no solvent is used. Preferably, the temperature in the case of guanidine bases such as TBD, for example, without solvent is above the melting point, but below 200° C., for example from 120° C. to 180° C., more preferably from 120° C. to 160° C., still more preferably from 130° C. to 140° C. The reaction times should be chosen accordingly. If a solvent is used, the reaction temperature and reaction time can be adapted. Via simple experimental series, those skilled in the art can establish the yield and the amount of byproduct, in particular unwanted isomers, and the experimental conditions can be adapted appropriately. The same applies to the use of other inventively usable catalysts. For instance, the phosphazene bases, depending on conditions, react even at 0° C., but from the energetic aspect, higher temperatures are preferred, for example room temperature. Those skilled in the art could accordingly replace the base selected, change the temperature or adapt the reaction time if too many byproducts occur. The amount of byproducts, in particular the trans/trans fatty acids, is assumed to be caused by a frequent change between deprotonation and protonation.

The process described for preparing the inventive conjugated octadecapolyenoic acids can be carried out continuously or batchwise in its different variants.

For the continuous procedure, a continuous-flow reactor, for example, can be used in which the catalyst capable of isomerization is present. The catalyst can be present not only as pure substance, but also immobilized, for example bound to a polymer (polymer-bound guanidine bases) (J. Mol. Catal. A: Chemical 109 (1996) 37-44; Pure Appl. Chem., A29(3), 249-261 (1992)); polymer-bound aminoiminophorane bases (Chimia 39 (1985) No.9, 269-272)) or enclosed in a support (cyclohexylguanidine in zeolite Y; THL, Vol. 38, No. 8, 1325-1328, 1997). Continuous reaction of vegetable oils with polymer-bound guanidine bases is described in BR 8202429.

The inventive formulation, in addition to the conjugated octadecapolyenoic acids and other unsaturated fatty acids described, can, in addition, comprise one or more additives.

The term “additives” is taken to mean other admixtures advantageous for nutrition or health, for example “nutrients” or “active ingredients”. The preparation can comprise one or more additives for animal or human nutrition or treatment and be diluted or mixed therewith. Additives can be administered together with or separately from the feedstuff, food, food supplement or drug. A food, food supplement, animal feed or drug preparation comprises no additives or no amount of additives which can be considered harmful for animal or human nutrition.

“Nutrients” are taken to mean those admixtures which are advantageous for the nutrition of humans or animals. Preferably, therefore, the inventive preparation also comprises:
vitamins, for example vitamins A, B₁, B₂, B₆, B₁₂, C, D₃, and/or E, K₃, folic acid, nicotinic acid,
taurin,
carboxylic acids and salts thereof, for example tricarboxylic acids, citrate, isocitrate, trans/cis-aconitate, and/or homocitrate,
enzymes, for example phytases,
carotenoids,
minerals, for example P, Ca, Mg, Mn and/or Fe,
proteins,
carbohydrates,
fats,
amino acids and/or
trace elements, for example Se.

The preparation can also comprise pyruvic acid, L-carnitine, lipoic acid, coenzyme Q10, aminocarboxylic acids, for example creatine.

“Active ingredients” are taken to mean those substances which support the use of the inventive formulation in pharmaceutical preparations or serve for their action of treating disease, in particular treatment of cancer, diabetes, AIDS, allergies and cardiovascular disorders.

Consequently, the inventive formulation can also comprise preservatives, antibiotics, antimicrobial admixtures, antioxidants, chelating agents, physiologically harmless salts etc.

The inventive formulations can also contain flavorings.

“Additives” are also taken to mean antioxidants. Antioxidants are advantageous, for example, for protecting the double bonds of the fatty acids against oxidation. However, the general health-promoting action of antioxidants is also known. For instance, in animal nutrition, as antioxidants, use is preferably made of ethoxyquin, ascorbic acid, t-butylated hydroxytoluene, t-butylated hydroxyamisol, ascorbyl palmitate, otherwise gamma- and alpha-tocopherols, tocotrienol, rosemary extract, isoflavones and carotenoids and naturally occurring polyphenols, for example flavonoids are also used.

The inventive formulation can comprise from 0.05 to 10% antioxidants. Preferably, it comprises from 0.1 to 6% antioxidant.

The inventive formulation can also comprise supports. For this, use can be made of, for example, customary inert supports. An “inert” support shall not exhibit any adverse interactions with the components used in the inventive formulation and must be safe for the use as aid in the respective uses, for example in foods, food supplements, feedstuffs, feed additives, pharmaceutical and cosmetic preparations.

Examples of suitable support materials which may be mentioned are: low-molecular-weight inorganic or organic compounds, and also relatively high molecular-weight organic compounds of natural or synthetic origin.

Examples of suitable low-molecular-weight inorganic supports are salts, such as sodium chloride, calcium carbonate, sodium sulfate and magnesium sulfate, or kieselguhr or silycic acids such as silicon dioxides or silica gels or silycic acid derivatives, for example silicates.

Examples of suitable organic supports are, in particular, sugars, for example glucose, fructose, sucrose, dextrins, starch products, in particular corn starch and cellulose preparations. Examples of further organic supports which may be mentioned are: corn cob flour, ground rice hulls, wheat semolina bran or cereal flours, for example wheat, rye, barley and oat flour or brans or mixtures thereof.

The support materials can be present in the inventive formulation, based on dry basis, in a content of from about 10 to 85% by weight, preferably from about 20 to 85% by weight.

In addition, the inventive formulation can comprise stabilizers, for example inorganic salts containing divalent cations. Examples of these are zinc sulfate, magnesium sulfate and calcium sulfate in a content of from about 0.1 to 10% by weight, preferably from about 0.5 to 5% by weight. Furthermore, further nutritionally relevant admixtures can be present, for example vitamins (for example vitamins A, B₁, B₂, B₆, B₁₂, D₃, E, K₃ and the like) or trace elements (for example manganese, iron, copper, zinc, iodine, selenium in the form of suitable salts). The total content of such admixtures can be for example in the range of from 1 to 10% by weight, based on the dry weight of the pulverulent formulation.

The inventive formulation can also comprise binders. Examples of suitable binders which may be mentioned are: solutions of carbohydrates, for example glucose, sucrose, dextrins and the like, sugar alcohols, for example mannitol, or polymer solutions, for exampole solutions of hydroxypropylmethylcellulose (HPMC), polyvinylpyrrolidone (PVP), ethoxylated cellulose (EC), ethylcellulose or propylcellulose. The binder content, based on the dry weight of the pulverulent formulation, can be in the range from about 0 to 20% by weight, for example from 1 to 6% by weight, depending on the type and adhesive properties of the binder used. For binding, in the simplest case, heat can also be used.

As already described above, the inventive solution consists in the fact that the formulations comprising the abovedescribed octadecapolyenoic acids are pulverulent. That is to say the inventive formulation can be present as adsorbate, beadlet, powder, granules, pellet, extrudate, agglomerate and/or as combinations thereof.

The inventive formulations can, in addition, be provided with a coating. This can serve, for example to improve the product properties, such as dusting behavior, flow properties, water absorption capacity, storage stability, protection of the active ingredient, delay or acceleration of the release of active ingredient, reinforcement of the mechanism of action or to achieve additional effects. For the coating, use can be made of, for example, fats, waxes, oils, biological and synthetic polymers.

The inventive pulverulent formulations preferably have a mean particle size of from 10 to 2000 μm. Particular preference is given to a mean particle size of from 20 to 800 μm.

Very particular preference is given from 80 to 600 μm, most preference to from 400 to 600 μm.

The inventive pulverulent formulations can be prepared in various ways.

Production of Adsorbates

For example, for production in the form of an adsorbate, one or more supports can be charged in a mixer or a fluidized-bed reactor and conjugated octadecapolyenoic acid or derivatives thereof, preferably conjugated octadecadienoic acid and also, if appropriate, other components, can be added. In rare cases, the use of stirred fixed beds or migrating beds is conceivable.

Preferably, use can be made of batch mixers. The support material is charged, if appropriate together with additives. Plowshares, blades, screws or the like ensure a more or less intensive product mixing. Classic examples are plowshare mixers, conical screw mixtures or like apparatuses. Alternatively mixing the product is possible via agitation of the entire vessel. Examples of this are tumble mixers, drum mixers or the like. Another possibility is the use of pneumatic mixers (see Ullmann's Encyclopedia of Industrial Chemistry, Sixth Edition, Mixing of Solids).

The active ingredient is metered/added, if appropriate together with additives, generally via devices for dropwise addition or spraying. Examples of these are lances, sprinkler heads, single-fluid or multiple-fluid nozzles, in rare cases rotating trickling devices or atomization devices. In the simplest case, addition is also possible locally as a concentrated jet. Alternatively, the active ingredient can be charged first in the mixer in order thereafter to add the support.

The active ingredient can be added at superatmospheric pressure, atmospheric pressure or at pressure lower than atmospheric, preferably at atmospheric pressure and superatmospheric pressure.

In isolated cases it can be advantageous to preheat the active ingredient (lowering of viscosity, changing the wetting properties), and also to supply or remove heat via the vessel wall and/or the mixing tools. In isolated cases it can be necessary to remove water vapors or solvent vapors.

To increase the loading of the support material and to minimize oxygen inclusions, it can be expedient to evacuate the mixer containing the support prior to addition of the active ingredient, and also if appropriate to blanket it with protective gas. Depending on the support, this is to be repeated several times.

Alternatively, continuous mixers are suitable. The active ingredients and hulls are added in this case preferably at different sites in the mixer.

Adsorbates can be produced batchwise or continuously in fluidized beds. The supports are agitated by the if appropriate hot fluidizing gas. Suitable fluidizing gas is air or else inert gas. In particular cases it is expedient to supply or remove heat via the vessel wall and/or via heat exchanger surfaces immersed in the fluidized bed. Suitable fluidized beds and the peripherals required are part of the prior art.

The active ingredients and additives are metered and if appropriate preheated batchwise or continuously via the abovedescribed devices which are known to those skilled in the art.

In particular cases, adsorbates can advantageously be produced by a combination of mixer and fluidized bed.

As support material for the production of the adsorbates, use can be made of the abovementioned materials, for example. In particular, salycic acids are suitable which, depending on the production process, are in fine or coarse form.

Production of Beadlets

Spray-formulated products can be produced, for example by, in a first step, preparing an aqueous solution of a protective colloid, preferably gelatin and/or gelatin derivatives and/or gelatin substitutes such as plant proteins, polysaccharides or modified starches, with addition of one or more materials selected from the group consisting of mono-, di- or polysaccharides, preferably corn starch, and, by adding antioxidants and the active ingredient (octadecapolyenoic acid) with stirring, a dispersion first forms, the aqueous solution of the colloid being the homogeneous phase of the dispersion.

As spraying aid, this can be made of, for example, a hydrophobic silica, corn starch or metal salts of higher fatty acids. It is also conceivable to use modified corn starch, hydrophilic silica, tricalcium phosphate and calcium silicates or mixtures of two or more of these substances. Likewise, mixtures of said fatty acids and silicas can also be used for the process. As metal salts of higher fatty acids having from 16 to 18 carbon atoms, use can be made of, for example, calcium or magnesium stearate.

The spraying aid can be introduced into the spray compartment above the fluidized bed with uniform distribution in from 0.01 to 0.25 times the amount by weight, based on the dispersion.

Colloids which can be used are preferably animal proteins, such as gelatin, for example from 50 to 250 Bloom, or casein.

The spraying aids are introduced directly into the spraying zone. The layer of the spraying aid which is produced during spraying stabilizes the particles to the extent that it prevents convergence of the particles during contact in the as yet unsolidified state. As a result it is possible to carry out direct drying on an adjoining fluidized-bed dryer.

The construction of the atomization unit does not have a critical effect on the product. For example, apparatuses can be used here as are described in EP0074050B1.

The spray-formulated products can be produced in a process variant by spraying the dispersion in a spray tower with co-use of a spraying aid and collection of the sprayed particles in a fluidized bed, a hydrophobic silica or the metal salt of a higher fatty acid, for example having from 16 to 18 carbon atoms, or mixtures with hydrophobic silica in from 0.02 to 0.15 times the amount by weight, based on the dispersion (and in the absence of significant amounts of other customary spraying aids such as starch powder), being introduced as spraying aid into the spraying compartment above the fluidized bed with uniform distribution at temperatures at which solidification of the if appropriate gelling colloid of the sprayed particles does not yet occur, collecting the spraying-aid-loaded particles, the colloidal mass of which is essentially not gelled, in a fluidized bed, and drying the particles in the fluidized bed in a manner known per se.

Colloids which can be used in the process described are preferably gelatin, for example from 70 to 200 Bloom, or casein. The amount of the colloid used is generally from 5 to 50% by weight, based on the end product, at water contents of the dispersion of from 30 to 70% by weight. To prepare the dispersion, the film-formers and subsequently the active ingredients are dispersed in the warm sugar solution at from 50 to 70° C. The dispersion is then atomized.

The construction of the atomization unit does not have a critical effect on the product. For example, nozzles or rapidly rotating atomizer discs can be used. The temperature of the dispersion to be atomized is likewise not a critical parameter. It is customarily from 60 to 90° C., which, with said colloids, gives viscosities of from 50 to 1200 mPas (60° C.). It is critical that at the time point of spraying, the particles come into contact with the hydrophobic spraying aid which is introduced in finely divided form directly into the spraying zone.

The great advantage of the proces is that the temperature in the spraying compartment need no longer be so low that gel formation of the active ingredient dispersion occurs, or that no longer need so much water be removed by large amounts of auxiliary powder that solidification of the droplets occurs. The process enables, for example, the spraying, at temperatures of from 25 to 30° C., of active ingredient dispersions which no longer solidify even at refrigerator temperatures (+4° C.). The amounts of the spraying aid necessary for this are in this case only from 0.02 to 0.15 times that of the dispersion.

The spray-formulated product, in a further process variant, can be produced by means of spray cooling. In this case a protective-colloid-containing dispersion, is sprayed, preferably by means of an atomization nozzle or atomization disc, at a temperature which is above the gel point of the emulsion, for example from 30° C. to 90° C., and at a viscosity of from preferably 50 to 600 mPas, in a spraying chamber in which the temperature is from 0° C. to 40° C., which produces microcapsules.

A spraying aid, for example corn starch or modified corn starch if appropriate in a mixture with other spraying aids, can be blown into the spraying chamber to prevent agglomeration of the gelatinized microcapsules and adhesion to the chamber walls. The spraying aid is preferably added in an amount of from 5 to 50%, measured on the weight of the end product.

The microcapsules can then be transferred to a fluidized bed in which they can be dried, if required, down to a residual water content of from 0 to 10% (preferably from 2 to 5%) and in which excess spraying aid is separated off. The temperature of the drying air is preferably from about 0° C. to about 60° C.

A further process variant for producing the spray-formulated products is the modified spray-drying process. This differs from spray cooling by the temperature in the spraying chamber being high, preferably from 50° C. to 95° C.

In the modified spray drying, the dispersion is sprayed into the spraying chamber, preferably at a temperature of from 5° C. to 99° C., and at a viscosity of from 50 to 600 mPas, by means of an atomization nozzle or an atomization disc.

A pulverulent spraying aid can be blown into the spraying chamber to prevent agglomeration of the gelatinized microcapsules and adhesion to the chamber walls. The spraying additive is preferably added in an amount of from 5 to 50%, measured on the weight of the end product.

The microcapsules can then be transferred into a fluidized bed in which they can be dried, if required, down to a residual water content of from 0 to 10% (preferably from 2 to 5%) and in which excess spraying aid is separated off. The temperature of the drying air is preferably from about 0° C. to about 60° C.

The spray-dried powders can also be produced in a manner such that, by changing the formula of the dispersion and the process, smaller particles are formed and spray-drying processes are employed.

In this case, for example in a first step, an aqueous solution of a protective colloid, preferably gelatin and/or gelatin derivatives and/or gelatin substitutes, is prepared with addition of one or more materials selected from the group consisting of mono-, di- or polysaccharides. By adding antioxidants and the active ingredient with stirring, first a dispersion is formed, the aqueous solution of the colloid being the homogeneous phase of the dispersion.

The dispersion can be sprayed, for example via single-fluid or two-fluid nozzles, or rotary atomizers, in a spraying tower, if appropriate with addition of powder-coating media or other additives.

The drying gas can be air or inert gas in a straight-through or recirculated gas procedure. The solids are removed in cyclones and/or filters. The spray drying is generally operated continuously.

By using spray dryers or sprayed fluidized beds, simple sprayed powders, loose agglomerates or compact agglomerates may be produced as required. The two last-mentioned embodiments are preferred over simple sprayed powders.

Production of Simple Sprayed Powders

In standard spray dryers powders are formed which are termed hereinafter simple sprayed powders. Standard spray dryers are characterized in that the hot drying gas is added at the sprayed-tower top, just as is the solution or dispersion to be atomized, in the spray tower heat exchange and mass transfer take place between gas and solution or dispersion and the gas and also the resultant simple sprayed powder is taken off at the bottom of the spray tower or at the side. This type of flow pattern is termed cocurrent flow. A countercurrent procedure is possible. The simple sprayed powder can be taken off at one or more points. A critical feature of the standard spray dryer is that there are no dust-recirculation systems or integrated fluidized beds.

The structure of simple sprayed powders may be described as spherical individual particles of differing particle size. In the case of unfavorable drying conditions, particles having cavities, or individual particles having an irregular surface can be formed.

Production of Loose Agglomerates

Said loose agglomerates have the structure of “raspberries”, that is to say they are generally formed from a plurality of sprayed individual particles which, as a result of the dust recirculation, grow together in the vicinity of the dust aggregate. Said loose agglomerates may be produced in spray dryers having dust recirculation with and without an integrated fluidized bed. Such apparatuses are offered in differing designs and are known to those skilled in the art.

Loose agglomerates may also be produced, for example, by, in a two-stage or multistage process, agglomerating simple sprayed powders in a fluidized bed or a mixer continuously or batchwise by spraying binder fluid or the abovedescribed solution or dispersion itself used in the production of the simple sprayed powders.

Compact agglomerates have a more or less “onion skin structure” and are convinced in fluidized beds by spraying the solution or dispersion onto preexisting particles. These spray-drying processes in fluidized beds may be operated batchwise or continuously. Those skilled in the art also term these processes, inter alia, spray-granulation processes. In the case of altered process parameters, in said fluidized-bed apparatuses, likewise loose agglomerates may be produced.

Production of Granules

Granules can be produced by charging into a mixer supports and/or spray-dried powders and also if appropriate additives and, by adding the active component and/or binder (preferably binder liquid—in the simplest case water) and/or additives, producing compact granules.

The mixer is preferably a blade mixer or plowshare mixer. The liquid components are added as described above (dropwise or sprayed), so that a pasty sticky phase is formed. By a suitable choice of the speed of rotation of the mixing tools and/or high-speed knife blades, the pasty phase is comminuted and compact granules are formed. Very large numbers are comminuted by mixing tools and knife blades and, on the other hand, fine powder are agglomerated.

The mode of operation is batchwise or continuous. Frequently, it is necessary to supply or remove heat via a heating jacket. The critical step is the combination of binder liquid, mechanical energy input via mixing tools and knife blades and establishing the required granulation time.

Coating layers can be added downstream in the mixer in the case of lower speed of rotation of the mixing tools and stationary knife blades or in a downstream mixer of related type.

The pasty sticky phase can also be shaped by pressing it through the matrix of an extruder. The process is characterized in that pressed rods are formed which if appropriate are redried and then coated.

Pulverulent formulations obtained in the process described can be further provided with a coating.

Coating materials which can be used are:
1) Fats, for example those
   of animal origin
   of plant origin
   of synthetic origin
2) Waxes, for example
   plant (for example candelilla wax, carnauba wax, rice germ oil wax etc.)
   animal (for example lanolin, beeswax, schellack, spermicetti)
   chemically modified (jojoba wax, sasol wax, montan ester wax)
3) animal proteins, such as gelatin, for example
   from cattle
   from pig
   from fish
4) plant proteins, such as
   soybean protein
modified starches
polysaccharides

In principle, other coatings from the solution are also conceivable, for example sugar coating. Likewise, use can be made of:
plant oils, for example sunflower, thistle, cottonseed, soybean, corn germ and olive oils, rapeseed, linseed, olive, coconut, oil palm kernel and oil palm oils
semisynthetic oils, for example medium-chain triglycerides or mineral oils
animal oils, for example herring, sardine and whale oils.

Examples of synthetic polymers usable as coatings are:
a) polyalkylene glycols, in particular polyethylene glycols, having a number-average molecular weight of from about 400 to 15 000, preferably from about 400 to 10 000;
b) polyalkylene oxide polymers or copolymers having a number-average molecular weight of from about 4000 to 20 000, preferably from about 7700 to 14 600; in particular block copolymers of polyoxyethylene and polyoxypropylene; for example Lutrols (trademark of BASF AG), for example
   F68 (block polymer polyoxyethylene polyoxypropylene)
   F127 (block polymer polyoxyethylene polyoxypropylene)
c) polyvinylpyrrolidone having a number-average molecular weight of from about 7000 to 1000 000, preferably from about 44 000 to 54 000;
d) vinylpyrrolidone/vinyl acetate copolymers having a number-average molecular weight of from about 30 000 to 100 000, preferably from about 45 000 to 70 000; for example Kollicoat SR (trademark of BASF AG)
e) poly(vinyl alcohol) having a number-average molecular weight of from about 10 000 to 200 000, preferably from about 20 000 to 100 000; and
f) hydroxypropylmethylcellulose having a number-average molecular weight of from about 6000 to 80 000, preferably from about 12 000 to 65 000.
g) alkyl (meth)acrylate polymers and copolymers having a number-average molecular weight of from about 100 000 to 1000 000; in particular ethyl acrylate/methyl methacrylate copolymers and methyl acrylate/ethyl acrylate copolymers, for example Kollicoat MAE (copolymer of methacrylic acid ethyl acrylate); and
h) poly(vinyl acetate) having a number-average molecular weight of from about 250 000 to 700 000, if appropriate stabilized with polyvinylpyrrolidone.
i) polyethylene
k) ethylcellulose
l) Kollicoat EMM (trademark of BASF AG)

For the coating, use can be made of, for example, a liquid which is as highly concentrated as possible and still sprayable, for example a from 1 to 50% strength by weight aqueous or nonaqueous solution or dispersion of one or more of said coating materials. Likewise, pulverulent coating materials can be used.

The coating can be applied in a similar manner to the abovedescribed processes for producing the adsorbates. That is to say the coating can be performed in the abovedescribed mixers or fluidized-bed apparatuses directly following the powder production (+CLA) or in a downstream process step. For instance, for example, the material to be coated (that is to say the octadecapolyenoic-acid-containing material) is charged into a fluidized-bed apparatus or a mixer and, with heating of the charge, the coating material is applied. The process can also be carried out without heating. In particular cases, it is necessary to the application of the coating, to add powder-coating materials such as talcum, silicates or the like to avoid sticking.

The coating material can be added at superatmospheric pressure, atmospheric pressure or at a reduced pressure compared with atmosphere, preferably at atmospheric pressure and reduced pressure.

In particular cases it can be advantageous to preheat the coating material (reducing viscosity, changing wetting properties), and also to supply or remove heat via the vessel wall and/or the mixing tools. In particular cases it is necessary to remove water vapor or solvent vapor.

To increase the loading of the particles and to minimize oxygen inclusions, it can be expedient to evacuate the material to be coated in the mixer prior to addition of the active ingredient, and also if appropriate to blanket it with protective gas. Depending on the material to be coated, this must be repeated several times.

According to a further variant of the production, the pulverulent formulation charged into a fluidized bed or mixer can be coated by a melt of the coating materials. For the coating, in this case, use can be made of in particular polyalkylene glycols, in particular polyethylene glycols, having a number-average molecular weight of from about 1000 to 15 000, preferably from about 1000 to 15 000; and poly(alkylene oxide) polymers or copolymers having a number-average molecular weight of from about 4000 to 20 000, in particular block copolymers of polyoxyethylene and polyoxypropylene.

As required, a little release agent can be added from time to time. Suitable release agents are, for example, pulverulent silicas, talcum, stearates and tricalcium phosphate.

The content of the coating mass of the coated formulation is 1-50% by weight, preferably 5-30% by weight, particularly preferably 8-20% by weight.

A further production variant is spray cooling. In this case the coated formulations can be obtained by dispersing the octadecapolyenoic-acid-containing mixtures and if appropriate other constituents and/or adjuncts in melts of suitable coating media and then atomizing and/or comminuting the resultant dispersions and solidifying them. Suitable coating media in the form of melts are materials whose melting point is above 30° C. Examples which may be mentioned are fats, waxes, oils, lipids, lipid-like and lipid-soluble substances having appropriate melting points.

These dispersions are then atomized in a cold gas stream, with and without the use of powder-coating materials, so that coated preparations comprising CLA are formed. These processes are known to those skilled in the art, for example under the names spray cooling, spray solidification, prilling or melt encapsulation, and also solidification on cooling belts, cooling rollers, tableting discs and tableting belts.

Preferably, the melts are produced in a first step before the octadecapolyenoic-acid-containing mixtures are added and dispersed. The dispersion can be performed batchwise in a stirred tank or else continuously in, for example, pumps which are suitable therefor or owing to sufficiently high turbulence simply in injections and piping. It is also possible to use static mixers, or orifice plates or the like. Protective heating of the required plant components, including the piping and atomizing elements, is known to those skilled in the art.

As cooling gas, use is preferably made of air and nitrogen. The gas can be conducted cocurrently, countercurrently or in cross flow. The process can be carried out in classic spray towers, prilling towers or other vessels. Fluidized beds with and without holdup are likewise suitable. The process can be operated batchwise or continuously. It is possible to separate off the solids, for example in cyclones or filters. Alternatively, collecting the solids, with or without subsequent cooling, in fluidized beds or mixers is conceivable.

Suitable atomizing elements are nozzles (single- and two-fluid nozzles or special types) and also atomizer wheels or atomizer discs or atomizer plates or atomizer baskets, or special types thereof.

In a further embodiment, the resultant dispersions are atomized and solidified in liquids in which neither the octadecapolyenoic-acid-containing mixtures nor the coating media are soluble. A classic solid-liquid separation with subsequent drying led to the inventive preparation.

The formulations described can be used in particular in foods, food supplements, feedstuffs, feed additives and also cosmetics or pharmaceutical preparations.

In the case of use as food, the inventive formulations are added in amounts of from 0.1 to 20% by weight. Particularly preferably, the foods comprise from 0.2 to 10% by weight of the formulation.

In foods, the inventive formulation can be combined with customary food components. These can comprise plant products or else animal products, in particular fatty foods such as butter or margarine; sugars, if appropriate in the form of syrups, fruit preparations, such as fruit juices, nectar, fruit pulps, purees or dried fruits; cereal products and also starches of said cereals; milk products, such as milk protein, whey, yogurt, lecithin and lactose.

In the case of use for food supplements, the inventive formulations are used in amounts of preferably 20-80% by weight, more preferably 30-70% by weight.

In the case of use as feedstuff and/or feed additive, preferably use can be made of amounts of from 20 to 80% by weight of the formulation. Particularly preferably, the feedstuff comprises from 55 to 75% by weight of the formulation. In the feedstuff and/or feed additive, adjuncts can also be present.

“Adjuncts” are taken to mean substances which improve the product properties, such as dust behavior, flow properties, water absorption capacity and storage stability. Adjuncts and/or mixtures thereof can be based on sugars, for example lactose or maltodextrins, based on cereal products or legume products, for example corn cob flour, wheat bran and soybean meal, based on mineral salts, inter alia, calcium salts, magnesium salts, sodium salts, potassium salts, and also D-pantothenic acid or salts thereof themselves (D-pantothenic acid salt prepared chemically or by fermentation).

In addition, the feedstuff and/or the feed additives can have components which comprise inactive, living and/or multiplying organisms producing contents of calendic acid or other additives. These can be, for example microorganisms, preferably fungi, yeasts and/or bacteria. The inventive feedstuff and/or the inventive feed additive can comprise, for example, inactive, living and/or multiplying contents of fungi of the genus Mucor, yeasts of the genus Saccharomyces and/or bacteria of the enterobacteriaceae, such as E. coli, salmonellae, such as Salmonella typhimurium, Proteus vulgaris, pseudomonads, such as Pseudomonas matophila, bacillaceae, such as Bacillus subtilis or Bacillus cereus, coryneform bacteria, such as Corynebacterium glutamicum or Brevibacterium breve and/or Actinum mycetalis and/or mixtures thereof.

If conjugated trans/cis octadecatrienoic acids, in particular calendic acid, are administered individually or in combination in the feedstuff, the active compounds are administered as pure substance or mixtures or as liquid or solid extracts together with customary feedstuff constituents. Examples of customary feedstuff constituents are: corn, barley, wheat, oats, rye, triticale, sorghum, rice and brans, semolina brans and flours of these cereal species, soybeans, soybean products such as soybean extraction meal, rapeseed, rapeseed extraction meal, cottonseed and extraction meal, sunflower seeds, sunflower extraction meal, linseed, linseed extraction meal, oilseed expeller meals, field bean and peas, gluten, gelatin, tapioca, yeasts, single cell protein, fish meal, salts, minerals, trace elements, vitamins, amino acids, oils/fats and the like.

The composition of feedstuff and/or feed additive should be such that the corresponding nutrient requirement is optimally covered for the respective animal species. Generally, plant feedstuff components are selected such as corn meal, wheat meal or barley meal, whole soybean meal, soybean extraction meal, linseed extraction meal, rapeseed extraction meal, grass meal or pea meal as crude protein sources. To ensure an appropriate energy content of feedstuff, soybean oil or other animal or vegetable fats are added. Since the plant protein sources comprise some essential amino acids only in an inadequate amount, feedstuffs are frequently enriched with amino acids. This concerns especially lysine and methionine. To ensure the mineral and vitamin supply of the farm animals, furthermore, minerals and vitamins are added. The type and amount of the minerals and vitamins added depends on the animal species. To cover the nutrient and energy requirement, complete diets can be used which contain all nutrients in a ratio to one another which covers requirements. It forms the sole feed of the animals. Alternatively, a supplementary feed can be added to a grain feed made of cereal. This relates to protein, mineral and vitamin-rich feed mixtures which expediently supplement the grain feed.

The use of the inventive formulations can, for example in the case of persons or animals weakened by disease, lead to a more rapid recovery. Use is advantageous, for instance, to achieve buildup of the body, for example after a relatively long disease which is accompanied by weight loss, for example chemotherapy, and to support or accelerate the recovery process.

In the case of use as pharmaceutical preparation, the inventive formulation can comprise other active ingredients. The active ingredients can serve for the treatment of cancer, cardiovascular disorders, for example arteriosclerosis, diabetes, allergies and the support of diets, or improve the action of the inventive preparation. A drug for the treatment of diabetes can comprise, for example, insulin, sulfonylureas, sulfonamides, lipoic acid, y-glucoxidase inhibitors, thiazolidinediones, mefformin and/or acetylsalicylic acid. Cancers are treated, for example, by adding cytostatics such as vinca alkaloids, alkylating agents, for example chlorambucil, melphalan, thio-TEPA, cyclophospamide, etc., by folic acid analogues, such as aminopterin or methotrexate, or by giving immunosuppressives, for example cyclophophosphamide and azathioprin, glucocorticoids, such as prednisolone or cyclosporin. HIV infections or AIDS can be treated, for example, by administering reverse-transcriptase inhibitors and/or protease inhibitors. Allergies are treated, for example, by stabilizing mast cells, for example by cromoglyxate, by blockading the histamine receptors, for example by H1 antihistamines, or by functional antagonists of the allergy mediators, for example by alpha-sympathomimetics, adrenalin, β2-sympathomimetics, theophylline, ipratropium or glucocorticoids. Cardiovascular disorders are treated using coagulation inhibitors, ACE inhibitors, cholesterol-lowering agents such as steatines and fibrates, niacins, cholestyramines.

In addition, the drug can comprise one of the abovementioned suitable additives.

Hereinafter the invention is described in more detail with reference to the examples:

### EXAMPLES 1-6

Preparation of Formulations Comprising CLA Esters

(Eight) different formulation processes were used:
1. Spray formulation (for production of beadlets)
2. Modified spray-drying process (for production of beadlets)
3. Adsorbate production
4. Coating of adsorbates
5. Spray drying
6. Spray solidification
7. Production of agglomerates/granules
8. Fixing of CLAs in adsorbates, using fats

### Example 1a

### Production of a Spray Formulation in a Pilot Plant

27.4 kg of water are heated to 50° C. 5.5 kg of gelatin (type A, 100 Bloom, DGF Stoess) are then added and allowed to swell with gentle stirring. After one hour, 5.3 kg of gelatin hydrolysate (Gelitasol PA®, DGF Stoess) and 4.0 kg of corn starch (KP 4076 from Cerestar) are added. After a further 10 minutes, 10.1 kg of a fatty acid methyl ester mixture (previously stabilized with 1% ethoxyquin, (Raluquin) from Raschig, based on the mixture) having a CLA methyl ester content of approximately 65% are added. By stirring with a propeller agitator an emulsion is produced.

The resultant emulsion is sprayed into a spraying tower at a temperature of 60° C. and a spraying pressure of 30 bar. During spraying, hydrophobic silica (Sipernat D 17®, Degussa) is blown into the spraying zone at rates of 10 kg/h. A moist dry powder is obtained which is then dried in a fluidized bed at 50° C. to a residual moisture of 4.5%.

The dry powder has the following particle size distribution:
D(v,0.1)=117 μm,
D(v,0.5)=189 μm,
D(v,0.9)=289 μm,
D[4,3]=196 μm.

### Examples 1b-1f

### Production of Spray Formulations in the Laboratory

On the basis of the procedure described in example 1a, a plurality of formula and process variations were carried out on a laboratory scale and powders were produced. Differences exist in the temperature during production of the emulsion (=60° C.), in the spray pressure (=4 bar) and in the redrying (room temperature, overnight). Samples were produced both on the basis of CLA ethyl ester and also examples based on CLA methyl ester:

| | Blade stirrer 500 | | | U-Turrax 10 000 | |
|---|---|---|---|---|---|
| | rpm | | | rpm | |
| | Examples | | | | |
| | 1 b | 1 c | 1 d | 1 e | 1 f |
| Feed materials | % | % | % | % | % |
| CLA-ME | | 40.0 | | | 40.0 |
| CLA-EE | 40.0 | | 40.0 | 40.0 | |
| EQ | 0.4 | 0.4 | 0.4 | 0.4 | 0.4 |
| Gelatin 100 Bloom A | 30.0 | 20.0 | 20.0 | 30.0 | 20.0 |
| Gelitasol PA | | 20.0 | 20.0 | | 20.0 |
| Corn starch | 22.0 | 14.0 | 14.0 | 22.0 | 14.0 |

All powders have a residual moisture after drying between one and two percent.

Using sample 1c, the particle size distribution is again determined:
D(v,0.1)=154 μm,
D(v,0.5)=206 μm,
D(v,0.9)=298 μm,
D[4,3]=216 μm.

### Example 2

### Production in the Modified Spray-Drying Process

2 kg of gelatin (Bloom value 240) and 2 kg of sucrose are dissolved at 65° C. in an emulsion tank in 6.0 liters of demineralized water and stored overnight to remove enclosed air bubbles. In a glass beaker, 2.4 kg of CLA oil are heated to 65° C. The oil is then added, with slow stirring, to the aqueous solution of gelatin and sucrose and then stirred vigorously for a further 30 minutes at 65° C. The resultant emulsion is diluted with 2 liters of demineralized water and adjusted to a viscosity of 160 cP. The mean particle size of the oil droplets is 0.4 μm.

Thereafter the emulsion is atomized in a spray tower, the droplets powder-coated with starch and dried by spray cooling. After screening (mesh 30/120) a yield of 6.75 kg of a particulate product having a total oil content of 29.8% is achieved

### Example 3a

### Production of an Adsorbate

700 g of a silica support (Sipernat 22®, Degussa) are charged into a plowshare mixer (Loedige type M5 GR). With stirring at approximately 200 rpm, the mixture of 720 g of CLA ethyl ester oil and 7.2 g of ethoxyquin is sprayed on via a spray pistol in the course of 4.5 min. The mixture is then further stirred for 8 min at an agitator speed of 345 rpm. This produces a homogeneous free-flowing powder.

### Example 3b-3d

### Production of Further Adsorbates

In the same mixer, with addition of CLA methyl ester oil onto two different silica supports (also Tixosil 38 X®, Rhodia), homogeneuos free-flowing powders are prepared in a similar manner.

| | | Formula in % | | | |
|---|---|---|---|---|---|
| | | | | | Residual |
| Examples | Silica | Silica | CLA-ME | EQ | moisture |
| 3.b | Sipernat 22 | 45.3 | 51.6 | 0.5 | 2.6 |
| 3.c | Tixosil 38x | 54.2 | 41.2 | 0.4 | 4.2 |
| 3.d | Tixosil 38x | 44.1 | 52.0 | 0.5 | 3.4 |

Sample 3d has the following particle size distribution:
D(v,0.1)=113 μm,
D(v,0.5)=240 μm,
D(v,0.9)=410 μm,
D[4,3]=252 μm.

### Example 4a

### Production of a Coated Adsorbate

500 g of a CLA ethyl ester adsorbate on Tixosil 68®, Rhodia, are charged into a laboratory fluidized bed (NIRO Aeromatic; type MP 1). The adsorbate contains 50% of a 61% strength CLA ethyl ester and also 0.5% ethoxyquin as stabilizer. The product is fluidized at room temperature (air rate approximately 30 m³/h) and a 55% strength solution of Diofan 193 D is sprayed onto the fluidized product in the course of 21 minutes. Then, at an inlet air temperature of 32-38° C. and at an air throughput of 25 m³h the product is dried. The drying time is 23 minutes. The product thereafter has a residual moisture content of 2.1% and has a theoretical content of 40% CLA ethyl ester (crude).

### Examples 4a-4e

### Production of Further Coated Adsorbates

Coating of CLA-EE adsorbate on Tixosil 68 (EE = ethyl ester)
Active ingredient content in the adsorbate (crude): 50%; stabilized with 1% ethoxyquin

| Example | | | | Composition of the |
|---|---|---|---|---|
| No. | Coating material | Type | Liquid phase | end product |
| 4a | Diofan 193 D | Copolymer | 55% strength | 40% CLA-EE (crude) |
| | from | dispersion based | dispersion in | 37.9% Tixosil 20% |
| | BASF | on vinylidene | water | Diofan 193D |
| | | chloride and | | 2.1% residual |
| | | methyl acrylate | | moisture |
| 4b | Lutrol E 6000 | Poly(ethylene | 40% strength | 40% CLA-EE (crude) |
| | from | glycol) | solution in water | 38.4% Tixosil 68 |
| | BASF | Mw: approx. | | 20% Lutrol E 6000 |
| | | 6000 | | 1.6% residual |
| | | | | moisture |
| 4c | Polygen WE 3 | Aqueous | 22% strength | 43% CLA-EE (crude) |
| | from | emulsion based | dispersion in | 41% Tixosil 68 |
| | BASF | on polyethylene | water | 12.6% Polygen WE 3 |
| | | wax | | 2% residual moisture |
| | | | | 1.4% Sipernat 22 S |
| 4d | Edenor NHTi-G | Hydrogenated | Melt (120° C.) | 40% CLA-EE (crude) |
| | from | beef tallow | | 38.4% Tixosil 68 |
| | Henkel KGaA | (Glycerine | | 20% NHTi-G |
| | | trioleate) | | 1.6% residual |
| | | | | moisture |
| 4e | Collicoat SR | Copolymer | 30% strength | 40% CLA-EE (crude) |
| | 30D | dispersion based | dispersion in | 37.6% Tixosil 68 |
| | from | on methacrylic | water | 20% Collicoat SR30D |
| | BASF | acid and ethyl | | 2.4% residual |
| | | acrylate | | moisture |

### Examples 4f-4m

### Production of Coated Adsorbates in the Lödige Mixer

1320 g of a CLA-ME adsorbate are charged into a preheated plowshare mixer (Loedige type M5 GR). The adsorbate is heated with a slow speed of rotation of 50 rpm. In parallel, a fat (for example Shoguwar 41) is melted in the glass beaker. Thereafter, 147 g of the fat are applied to the adsorbates from the glass beaker by simple pouring at a speed of 110-150 rpm of the mixer. With stirring, the product is cooled in the mixer to approximately 30° C. and the adsorbate thus encased is discharged and packaged.

| | | Formula in % | | | | |
|---|---|---|---|---|---|---|
| Exam- | Coating | | CLA- | | | Residual |
| ples | material | Tix.38X | ME | EQ | Coating | moisture |
| 4f | Lanolin | 35.3 | 52.1 | 0.5 | 10.3 | 1.8 |
| | from Fluka | | | | | |
| 4g | Shoguwar 41 | 35.2 | 51.8 | 0.5 | 10.3 | 2.2 |
| | from Arhus Olie | | | | | |
| 4h | Cutina CP | 35.2 | 51.9 | 0.5 | 10.3 | 2.1 |
| | from Henkel | | | | | |
| 4i | Japan wax | 35.2 | 51.9 | 0.5 | 10.3 | 2.1 |
| | from Kahl | | | | | |
| 4j | Edenor NHTI-G | 35.4 | 52.1 | 0.5 | 10.3 | 1.6 |
| | from Henkel | | | | | |
| 4k | Beeswax | 35.3 | 52.1 | 0.5 | 10.3 | 1.5 |
| | from Kremer- | | | | | |
| | Pigmente | | | | | |
| 4l | Candelilla wax | 35.4 | 52.2 | 0.5 | 10.3 | 1.6 |
| | from Kremer- | | | | | |
| | Pigmente | | | | | |
| 4m | Schellack wax | 35.5 | 52.4 | 0.5 | 10.3 | 1.3 |
| | from Kremer- | | | | | |
| | Pigmente | | | | | |

The powder of example 4g has the following particle size distribution:
D(v,0.1)=153 μm,
D(v,0.5)=266 μm,
D(v,0.9)=415 μm,
D[4,3]=275 μm.

The powder of example 4k has the following particle size distribution:
D(v,0.1)=184 μm,
D(v,0.5)=302 μm,
D(v,0.9)=452 μm,
D[4,3]=311 μm.

### Examples 4n-4t

### Subsequent Application of Oils to the Adsorbates in the Lödige Mixer

1470 g of a CLA-ME adsorbate are charged into a plowshare mixer (Loedige type M5 GR). Thereafter, 163 g of an oil are applied to the adsorbates using a spray pistol at a speed of rotation of 200 rpm of the mixer. The product is then further stirred for 5 minutes at 345 rpm and the adsorbate thus encased is discharged and packaged.

| | | Formula in % | | | | |
|---|---|---|---|---|---|---|
| Exam- | | | | | | Residual |
| ples | Oil used | Tix 38 X | CLA-ME | EQ | Oil | moisture |
| 4n | Thistle oil | 34.0 | 52.2 | 0.5 | 10.4 | 2.9 |
| 4o | Olive oil | 34.0 | 52.3 | 0.5 | 10.5 | 2.7 |
| 4p | Beet oil | 34.0 | 52.3 | 0.5 | 10.5 | 2.7 |
| 4q | Sunflower oil | 33.9 | 52.3 | 0.5 | 10.5 | 2.8 |
| 4r | Peanut oil | 33.9 | 52.2 | 0.5 | 10.4 | 3.0 |
| 4s | Cottonseed oil | 34.0 | 52.2 | 0.5 | 10.4 | 2.9 |
| 4t | Corn germ oil | 33.9 | 52.3 | 0.5 | 10.5 | 2.8 |

The powder of example 4q has the following particle size distribution:
D(v,0.1)=112 μm,
D(v,0.5)=219 μm,
D(v,0.9)=371 μm,
D[4,3]=231 μm.

### Example 5

### Production of Granules in the Stirred Flask

100 g of the spray-dried powder produced in example 5 are placed in a 1000 ml four-necked flask. A 45% strength aqueous sucrose solution is added uniformly dropwise as binder liquid with stirring. The speed of rotation of the blade agitator is 500 rpm. In total 15 g of sucrose solution are added dropwise.

The fine spray-dried powder gradually agglomerates and agglomerates/pellets having a very broad particle size distribution from 100 μm to 3000 μm are formed.

The granules are further dried for one hour at 35° C. in a laboratory fluidized-bed reactor (Niro Aeromatic, MP1). Of the usable fraction, particles from 500 μm to 1000 μm are separated off and this gives 65 g.

### Examples 6-9

### Test of Stability

Experiments Carried Out:

A) Characterization of the formulation

B) Stability of CLA in said formulations

C) Tabletability of the formulation

D) Stability of CLA in tablets

Production of 4 Test Formulations (at Least 500 g in Each Case):

1. CLA beadlet (40%; example 6)

2. CLA beadlet (25%; example 7)

CLA adsorbate (uncoated 50%; example 8)

CLA oil (90%; example 9)

### Formulation of CLA for Tableting Experiments

Content: 90% CLA ethyl ester; stabilized with 1.0% DL-alpha-tocopherol

| Example | | |
|---|---|---|
| No. | Type | Composition |
| 6 | Spray-formulated dry powder | 40% CLA ethyl ester |
| | having hydrophobic silica as | 0.4% DL-alpha-tocopherol |
| | powder-coating material | 30% gelatin 100 Bl A |
| | | 22% corn starch |
| | | (residual moisture 2.1% |
| | | not determined: Sipermat |
| | | D 17) |
| 7 | Spray-cooled dry powder having | 28.7% CLA ethyl ester |
| | corn starch/TCP as | 0.3% DL-alpha-tocopherol |
| | powder-coating material | 22% gelatin 240 BL A |
| | | 22% sucrose |
| | | 25% corn starch |
| | | 1% tricalcium phosphate |
| | | (residual moisture: 3.6%) |
| 8 | Adsorbate on silica (Sipernat 22) | 50% CLA ethyl ester |
| | | 0.5% DL-alpha-tocopherol |
| | | 47.2% Sipermat 22 |
| | | (residual moisture 2.3%) |

A) Characterization of the Formulations

| | | Bulk | Tamped | Flow | Viscosity | PCD |
|---|---|---|---|---|---|---|
| Exam- | | density | density | property | [mPas | (d0.5) |
| ple | | [g/ml] | [g/ml] | [°/sec] | at 11.9° C.] | [μm] |
| 6 | CLA | 0.57 | 0.64 | 27/6.3 | — | 271 |
| | beadlet | | | | | |
| | 40% | | | | | |
| 7 | CLA | 0.60 | 0.69 | 26/6.8 | — | 250 |
| | beadlet | | | | | |
| | 25% | | | | | |
| 8 | CLA | 0.48 | 0.62 | 31/17.8 | — | 127 |
| | adsorbate | | | | | |
| | 50% | | | | | |
| 9 | CLA oil | — | — | — | 33.4 | — |

B) Stability of CLA in the Formulations

˜50 g for each formulation and storage condition were stored in an open twist-off glass.

Storage Conditions:

25° C./65% rH.

40° C./75% rH.

60° C.

The relative decrease of CLA in the individual formulations is measured at the following time points:

12 and 24 weeks
Stability at 20° C./65% rH

| | |
|---|---|
| | Content of CLA (%) |
| Formulation | after |

| according to | | 12 weeks' | 24 weeks' |
|---|---|---|---|
| example | | storage | storage |
| 6 | CLA beadlet | 98.9 | 87.0 |
| | 40% | | |
| 7 | CLA beadlet | 96.6 | 95.0 |
| | 25% | | |
| 8 | CLA | 87.8 | 15.0 |
| | adsorbate | | |
| | 50% | | |
| | (uncoated) | | |

Stability at 40° C./70% rH

| | |
|---|---|
| | Content of CLA (%) |
| Formulation | after rH |

| according to | | 12 weeks' | 24 weeks' |
|---|---|---|---|
| example | | storage | storage |
| 6 | CLA beadlet | 56.5 | 2 |
| | 40% | | |
| 7 | CLA beadlet | 92.9 | 2 |
| | 25% | | |
| 8 | CLA adsorbate | 0 | 0 |
| | 50% | | |
| | (uncoated) | | |

Stability at 60° C.

| | |
|---|---|
| | Content of CLA (%) |
| Formulation | after |

| according to | | 12 weeks' | 24 weeks' |
|---|---|---|---|
| example | | storage | storage |
| 6 | CLA beadlet | 45.2 | 25.0 |
| | 40% | | |
| 7 | CLA beadlet | 84.5 | 79.0 |
| | 25% | | |
| 8 | CLA adsorbate | 0 | 0 |
| | 50% | | |
| | (uncoated) | | |

Stability of CLA oil (example 9)

Content of CLA (%)
after

| | 12 weeks' | 24 weeks' |
|---|---|---|
| | storage | storage |
| At 40° C./70% rH | 0 | 0 |
| At 60° C./ | 0 | 0 |

C) Tableting Ability and Stability of CLA in tablets
Aids:

Kollidon C L, from BASF, Lot. No.: 91-1297

Kollidon V A 64, from BASF, Lot. No.: 07-4919

Aerosil 200, from Degussa

Avicel P H 102, from FMC, Batch 7929C

Mg stearate, from Bärlöcher, Batch MF 19-80519

Apparatus:

Eccentric tableting press, Pfrengle funnel, top-pan balance,

Turbula mixer, 0.8 mm screen

Tablet formula: Content 200 mg of CLA/Tabl.

| | Example | | |
|---|---|---|---|
| | 6 | 7 | 8 |
| | [g] | [g] | [g] |
| CLA formulation | 50.0 | 80.0 | 40.0 |
| Avicel | 53.8 | 23.8 | 63.8 |
| Aerosil | 2.4 | 2.4 | 2.4 |
| Kollidon VA 64 | 9.0 | 9.0 | 9.0 |
| Kollidon CL | 3.6 | 3.6 | 3.6 |
| Mg stearate | 1.2 | 1.2 | 1.2 |
| | | 120 | |

Production of the Tablets:

Prepare the tableting mixture without CLA, mix briefly and pass through a 0.8 mm screen. In each case add CLA and homogeneously mix each mixture for 10 minutes in the Turbula mixer.

Compress the finished batches on the tableting press with 20 mm stamp, facet (weight 1200 mg) at a compression force of 15 (8), 30 (6) and 30 (7) kN.

Characterization of the tablets:

| | Weight | Hardness |
|---|---|---|
| Formulation | [mg] | [N] |
| 6) | 1206 | 40 |
| 7) | 1197 | 40 |
| 8) | 1170 | 40 |

Stability of CLA in Tablets:

30 tablets per formulation and storage condition are stored in an open twist-off glass.

Storage Conditions:

25° C./65% R.H.

40° C./75% R.H.

The relative decrease in CLA in the individual formulations is measured at the following time points:

12 and 24 weeks

Tableting Behavior:

Tablets can be produced from all samples without problems.

Owing to the high content of sample powder, the tablet does not achieve high tablet hardness. Therefore, in order to obtain an acceptable tablet hardness, the compression force had to be varied in the various formulations.

Stability at 20° C./65% RH:

The measurement showed that the CLA content in the case of CLA beadlet 40%, CLA beadlet 25% and CLA adsorbate 50% (uncoated) remained virtually constant even after 24 weeks.

Stability at 40° C./70% RH

| | |
|---|---|
| | Content of CLA (%) |
| Formulation | after |

| according to | | 12 weeks' | 24 weeks' |
|---|---|---|---|
| example | | storage | storage |
| 6 | CLA beadlet | 73.9 | 0 |
| | 40% | | |
| 7 | CLA beadlet | 97.2 | 2 |
| | 25% | | |
| 8 | CLA adsorbate | 10.6 | 0 |
| | 50% | | |
| | (uncoated) | | |

1. A pulverulent formulation for the use in foods, food supplements, feedstuffs, feed additives, pharmaceutical and cosmetic preparations comprising at least one conjugated octadecapolyenoic acid characterized in [that less than 5% of the fatty acid content is 11-, 13-octadecadienoic acid isomers 8-, 10-octadecadienoic acid isomers, cis-, cis-octadecadienoic acid isomers or trans-, trans-octadecadienoic acid isomers or mixtures of these isomers].

2. A formulation according to claim 1, characterized in that less than 3% of the fatty acid content is 11-, 13-octadecadienoic acid isomers, 8-, 10-octadecadienoic acid isomers, cis-, cis-octadecadienoic acid isomers or trans-, trans-octadecadienoic acid isomers or mixtures of these isomers.

3. A formulation according to either of claims 1 or 2 characterized in that less than 1% of the fatty acid content is 11-, 13-octadecadienoic acid isomers, 8-, 10-octadecadienoic acid isomers, cis-, cis-octadecadienoic acid isomers or trans-, trans-octadecadienoic acid isomers or mixtures of these isomers.

4. A formulation according to one of claims 1-3, characterized in that it comprises octadecadienoic acid, octadecatrienoic acid or combinations of these octadecapolyenoic acids.

5. A formulation according to one of claims 1 to 4, characterized in that at least each 50% of the fatty acid content is 9-cis-, 11-trans- and 10-trans-, 12-cis-octadecadienoic acids.

6. A formulation according to one of claims 1 to 5, characterized in that at least 60% of the fatty acid content is 9-cis-, 11-trans- and 10-trans-, 12-cis-octadecadienoic acids.

7. A formulation according to one of claims 1 to 6, characterized in that at least 70% of the fatty acid content is 9-cis, 11-trans- and 10-trans-, 12-cis octadecadienoic acids.

8. A formulation according to one of claims 1 to 7 characterized in that, as octadecatrienoic acid, it comprises calendic acid or punicic acid.

9. A formulation according to one of claims 1 to 8, characterized in that it comprises stabilizers, antioxidants, supports or mixtures of these materials.

10. A formulation according to one of claims 1 to 9, characterized in that it has a mean particle size of from 20 to 800 μm.

11. A formulation according to one of claims 1 to 10, characterized in that it has a mean particle size of from 50 to 700 μm

12. A formulation according to one of claims 1 to 11, characterized in that it has a mean particle size of from 80 to 600 μm.

13. A formulation according to one of claims 1 to 12, characterized in that it has a mean particle size of from 400 to 600 μm.

14. A formulation according to one of claims 1 to 13, characterized in that it is present as adsorbate, spray-formulated product, granules or extrudate.

15. A formulation according to one of claims 1 to 14, characterized in that it is provided with a coating.

16. A formulation according to claim 15, characterized in that the coating comprises fats, waxes, animal proteins, plant proteins, vegetable oils, animal oils, semisynthetic oils or synthetic polymers or mixtures of one or more of these materials.

17. A process for producing an adsorbate according to claim 14, characterized in that a support or plurality of supports are charged into a mixer or fluidized-bed reactor and then conjugated, if appropriate preheated, octadecapolyenoic acid or derivatives thereof and also, if appropriate further components are added.

18. A process for preparing spray-formulated products according to claim 14, characterized in that they are obtained by dispersing octadecapolyenoic acids and, if appropriate other components in an aqueous solution of a film-forming protective colloid, spraying the dispersion in a spraying tower if appropriate with co-use of a spraying aid and collecting the sprayed particles and, if appropriate redrying them in a fluidized bed.

19. A process for producing spray-formulated products according to claim 14, characterized in that simple sprayed powders are prepared and these are agglomerated in a fluidized bed or a mixer continuously or batchwise by spraying on binder or a solution or dispersion of the formulation according to one of claims 1-13.

20. A process for producing granules according to claim 14, characterized in that supports or spray-dried products or mixtures of these substances and conjugated octadecapolyenoic acid are charged in a mixer and also, by addition of further octadecapolyenoic acid or binders or mixtures of these substances, the granules are produced.

21. A process for producing extrudates according to claim 14, characterized in that the supports or spray-dried products or mixtures of these substances and/or conjugated octadecapolyenoic acid are charged, mixed, and extrudates or pellets are shaped from the resultant pasty mass by means of an extruder or a pelleting press.

22. A process for producing a formulation according to one of claims 1-16, characterized in that the formulation is coated.

23. A process according to claim 22, characterized in that the coating is applied in a mixer or in a fluidized-bed reactor.

24. Food, food supplement, feedstuff, animal feed additive, cosmetic or pharmaceutical preparation comprising a formulation according to one of claims 1 to 16.

25. A food comprising from 0.1 to 20% by weight of the formulation according to claims 1 to 16.

26. A food comprising from 0.2 to 10% by weight of the formulation according to claims 1 to 16.

27. A food comprising from 0.2 to 1% by weight of the formulation according to claims 1 to 12

28. A food supplement comprising 10-90% by weight of the formulation according to claims 1-16

29. A food supplement comprising 20-80% by weight of the formulation according to claims 1 to 16.

30. A food supplement comprising 30-70% by weight of the formulation according to claims 1 to 16.

31. A feed additive comprising from 20 to 80% by weight of the formulation according to claims 1 to 16.

32. A feed additive comprising from 55 to 75% by weight of the formulation according to claims 1 to 16.

33. The use of the formulation according to one of claims 1 to 16 as feedstuff, feed additive, food, food supplement, cosmetic or pharmaceutical preparation.
